(19) United States
(12) Patent Application Publication
(10) Pub. No.: US 2026/0272581 A1
(43) Pub. Date: Sep. 17, 2026

(54) SURGICAL SYSTEM

(71) Applicant: MINMAXMEDICAL, SAINT-MARTIN-D'HÈRES (FR)

(72) Inventors: Jérémy QUINTIN, SAINT-MARTIN-D'HÈRES (FR); Sylvain FONTAINE, SAINT-MARTIN-D'HÈRES (FR); Elie FOURNIER, SAINT-MARTIN-D'HÈRES (FR); Thomas LONJARET, SAINT-MARTIN-D'HÈRES (FR); Cyrill MARCOL, SAINT-MARTIN-D'HÈRES (FR); Stéphane LAVALLEE, SAINT-MARTIN-D'HÈRES (FR)

(21) Appl. No.: 19/325,801

(22) Filed: Sep. 11, 2025

Related U.S. Application Data
(63) Continuation-in-part of application No. 19/162,629, filed on Mar. 8, 2024.

(30) Foreign Application Priority Data
Mar. 8, 2023 (EP) 23305312.3

(51) Int. Cl. A61B 34/32; A61B 34/10; A61B 34/20; A61B 34/30

(52) U.S. Cl. CPC A61B 34/32; A61B 2034/107; A61B 2034/2059; A61B 2034/305

(57) ABSTRACT

A surgical system comprises:
a robotic arm comprising, from a proximal to a distal end:
   a SCARA comprising a plurality of parallel motorized joints connected by a plurality of links extending in a horizontal plane, wherein, for each pair of links, a distal link is above a proximal link;
   an intermediate arm comprising four revolute motorized joints having a parallel-perpendicular-perpendicular-parallel relationship; and
   a wrist comprising an interface for holding a surgical tool, including three revolute motorized joints having a perpendicular-perpendicular-perpendicular relationship such that axes of said three revolute motorized joints are concurrent;
at least one control device configured to receive commands from a user; and
a control unit coupled to the robotic arm and the control device to operate the robotic arm according to at least a hand-guiding mode and a patient servoing mode.

The control unit is configured to:
determine a current configuration of the joints of the robotic arm;
compute a target configuration of the joints of the robotic arm to move the surgical tool based on commands received from the user and on the current configuration of the joints;
compute a trajectory of the robotic arm to achieve said computed target configuration; and
send instructions to at least one motor of the joints of the SCARA, the intermediate arm or the wrist to move the surgical tool according to the computed trajectory.

### CROSS REFERENCE TO RELATED APPLICATIONS

This application is a Continuation-in-Part of U.S. National Phase Application Filed Sep. 8, 2025, and assigned application Ser. No. 19/162,629, which application is a 35 U.S.C. § 371 national stage application of PCT Application No. PCT/EP2024/056265, filed Mar. 8, 2024, which application claims the benefit of European Application No. EP 23305312.3 filed Mar. 8, 2023, all of which are hereby incorporated by reference herein in their entireties.

### TECHNICAL FIELD

The present invention concerns the domain of surgical systems, and especially, it concerns a surgical system adapted to be used for orthopedic surgeries.

### TECHNICAL BACKGROUND

During the last few decades, the field of surgical systems has significantly grown, and especially surgical systems which are adapted to be used in computer-assisted medical intervention (CAMI), as referred to in the publication of S. Lavallée & P. Cinquin: “Computer assisted medical interventions” In K. H. Hohne, editor, NATO ARW, 30 Imaging in Medicine, Vol F60, 301-312, Berlin, June 1990. Springer-Verlag. Such systems aim to help surgeons in performing securer operations while also improving the accuracy and reproducibility of said operations. The use of those surgical systems also aims at improving the precision of surgical movements, lower invasiveness and in reducing the number and the seriousness of errors that can otherwise occur during those operations. Robotic aid to clinicians for execution of optimal surgery was introduced in the early 1980's in neurosurgery application. Since the 1990's, several assistive technologies combining part or all of 2D/3D imaging, navigation and robotics were developed with the primary focus of improving accuracy of surgical procedures, in view of improving clinical and functional outcomes for the patients.

A first generation of robots were developed as passive-robots. These passive-robots can for example consist of optical localizer or motorless encoded arms and they are particularly well-suited for navigation, but their use is difficult for executing complex surgical strategies. However, these passive-robots can be useful for performing simple operation such as ones wherein the movements needed are all about one single axis. For instance, the robot Cirq® developed by BrainLab is one of those passive robots.

A second generation of robots were developed as active robots. These active robots are designed to perform at least part of an intervention on their own from a planned procedure, i.e., without any real-time guidance from the surgeon, nor from any other operator. For instance, Robodoc® is an active robot developed by Integrated Surgical Systems, commercialized at the end of the 90's and adapted to perform part of hip replacements surgeries. Such active robots are generally accurate but raise safety and ergonomics issues.

Nowadays, many medical device suppliers are developing collaborative robots, i.e., robots with which the operators are able to cooperate. These collaborative robots are equipped with an input device through which the user of the system can ask for a displacement of the robot, and the system is then adapted to treat the received input and to displace the robot literally as asked by the user or to forbid the displacement asked, i.e. taking into account both the demand of the user and, for instance, safety parameter. One major drawback of those collaborative robots is that they are particularly adapted to be used for one dedicated kind of surgery, thus forcing the users to buy several robots if they want to be able to perform different surgeries.

One solution to increase the number of surgeries which can be realized with the help of one unique collaborative robot is to increase the number of degrees of freedom of such collaborative robots. One major issue of this increasing is that the robots become more and more complicated to drive for the user, especially when said collaborative robots are equipped with a unique input device.

### SUMMARY OF THE DISCLOSURE

Thus, there remains a need to provide a collaborative surgical system which can be used for several kind of surgeries, while being easy to drive for its user and safe both for the patient to be treated and for the surgeon and all the caregivers which can be in the surrounding of said system during surgical interventions.

The present invention falls in this context and aims at providing such a collaborative system.

An object of the present invention thus relates to a surgical system comprising:
a robotic arm comprising, from a proximal to a distal end:
   a SCARA comprising a plurality of parallel motorized joints connected by a plurality of links extending in a horizontal plane, wherein, for each pair of links, a distal link is above a proximal link;
   an intermediate arm comprising four revolute motorized joints having a parallel-perpendicular-perpendicular-parallel relationship; and
   a wrist comprising an interface for holding a surgical tool, including three revolute motorized joints having a perpendicular-perpendicular-perpendicular relationship such that axes of said three revolute motorized joints are concurrent;
at least one control device configured to receive commands from a user; and
a control unit coupled to the robotic arm and the control device to operate the robotic arm according to at least a hand-guiding mode and a patient servoing mode, the control unit being configured to:
determine a current configuration of the joints of the robotic arm;
compute a target configuration of the joints of the robotic arm to move the surgical tool based on commands received from the user and on the current configuration of the joints;
compute a trajectory of the robotic arm to achieve said computed target configuration; and
send instructions to at least one motor of the joints of the SCARA, the intermediate arm or the wrist to move the surgical tool according to the computed trajectory.

In some embodiments, the surgical system further comprises a tracking system coupled to the control unit, the tracking system being configured to track a pose of the surgical tool with respect to at least one region of interest of an anatomical structure to be treated by the surgical tool.

In some embodiments, the control unit comprises a storage unit storing a surgical plan defining the at least one region of interest to be treated and at least one initial pose of the surgical tool allowing a user of the system to start treating the region of interest of an anatomical structure.

In some embodiments, the control unit is configured to operate the robotic arm in an alignment mode, wherein the control unit is configured to
compute a target configuration of the joints of the robotic arm to move the surgical tool to the at least one initial pose;
compute a trajectory of the robotic arm to achieve said computed target configuration; and
send instructions to at least one motor of the joints of the SCARA, the intermediate arm or the wrist to move the surgical tool according to the computed trajectory.

In some embodiments, each motorized joint of the robotic arm comprises at least one encoder coupled to the control unit, the control unit being configured to determine the current configuration of the robotic arm based on information received from each encoder.

In some embodiments, the motorized joints of the intermediate arm and the wrist are arranged to have an analytical inverse kinematic solution, whereby the control unit is configured to calculate a set of angular positions for the joints for a desired pose of the surgical tool.

In some embodiments, the control unit is configured to identify a risk of singularity in a computed configuration of the robotic arm and determine at least one alternative configuration of the intermediate arm or the wrist to achieve a configuration of the robotic arm avoiding the singularity.

In the patient servoing mode, the control unit may be configured to update the trajectory of the robotic arm to maintain alignment of the surgical tool with the region of interest according to the surgical plan.

In the patient servoing mode, the control unit may be configured to send instructions to the joints of the SCARA to maintain their current position or lock the joints of the SCARA by stopping feeding the motors of the joints of the SCARA with an electrical current and activate brakes.

In some embodiments, the control device is fixed to the robotic arm.

In other embodiments, the control device is detachably mounted on the robotic arm.

Another object of the disclosure is a surgical system configured to treat a region of interest of an anatomical structure, the surgical system comprising
at least one robotic arm coupled with a surgical tool configured to treat the region of interest, the robotic arm comprising several links, two adjacent links being linked to one another by one motorized joint, the robotic arm comprising at least a first part with at least three degrees of freedom and a second part with at least six degrees of freedom, an end of the first part forming a base of the second part and the first part comprising a selective compliance assembly robot arm,
at least one control device configured to receive commands from a user of the system, and
at least one data processor,
   the surgical system being operable, at least, according to a hand-guiding mode, the data processor being configured to, as long as the hand-guiding mode is enabled:
determine current dynamics and kinematics of the robotic arm,
receive at least one user command from the control device,
determine, based on the received user command(s) and on the current dynamics and kinematics of the robotic arm, an intended displacement of the surgical tool,
determine at least one optimal trajectory of the robotic arm permitting to obtain the determined intended displacement of the surgical tool, while satisfying at least one mathematical function which defines authorized trajectories of the robotic arm,
compute and send at least one instruction(s) to at least one of the motorized joint(s) of the robotic arm, the execution of the at least one instruction(s) resulting in a displacement of the robotic arm according to the optimal trajectory,
   wherein when several optimal trajectories of the robotic arm are determined, the data processor is configured to select one of these optimal trajectories so that the displacement of the robotic arm implies the smallest number of movements of the motorized joint(s) of the first part possible.

The invention can be complemented by the following features taken alone or in combination:

The control device comprises at least one force and/or torque sensor and the control device is configured to, as long as the hand-guiding mode is enabled:
measure forces and/or torques applied by the user on the control device,
determine the at least one user command based on the measured forces and/or torques,
send the user command to the data processor.

The data processor is configured to detect and/or measure a force and/or torque applied on at least one of the motorized joints of the second part of the robotic arm, and the data processor is further configured to, as long as the hand-guiding mode is enabled:
determine, based on the detected and/or measured force and/or torque applied on the concerned motorized joint(s), a configuration of the concerned motorized joint(s) intended by the user,
select the optimal trajectory of the robotic arm so that the displacement of the concerned motorized joint(s) matches the intended configuration of said motorized joint(s).

The surgical system is associated with at least one tracking system configured to determine, at a predefined frequency, relative pose of the surgical tool with respect to the anatomical structure and with respect to the region of interest and to send a corresponding information to the data processor.

To determine the pose of the surgical tool with respect to the region of interest, the tracking system can for instance be configured to first determine the relative pose of the surgical tool with respect to the anatomical structure. Then, as the pose of the region of interest with respect to the anatomical structure is known, the tracking system is configured to determine the relative pose of the surgical tool with respect to the region of interest, based on the determined pose of the surgical tool with respect to the anatomical structure and on the known pose of the region of interest with respect to the anatomical structure. Alternatively, the tracking system can be configured to determine the relative pose of the surgical tool with respect to the anatomical structure and to send a corresponding information to the data processor, the data processor then being configured to determine the pose of the surgical tool with respect to the region of interest.

The tracking system can for instance be an optical tracking system, an electromagnetic tracking system, an inertial tracking system or a combination thereof.

The surgical system comprises at least one storage unit storing a surgical plan defining, at least one initial pose of the surgical tool allowing a user of the system to start treating the region of interest, wherein the surgical system is operable according to a pre-alignment mode and wherein the data processor is configured to, as long as the pre-alignment mode is enabled:
determine a current configuration of the robotic arm,
determine, based on the stored surgical plan and on the determined current configuration of the robotic arm, a displacement of the surgical tool needed to reach its initial pose,
determine at least one optimal trajectory of the robotic arm permitting to obtain the determined displacement of the surgical tool, while satisfying at least one mathematical function which defines authorized trajectories of the robotic arm,
compute and send at least one instruction(s) to at least one of the motorized joint(s) of the robotic arm, the execution of the at least one instruction(s) resulting in a displacement of the robotic arm according to the optimal trajectory,
   wherein when several optimal trajectories of the robotic arm are determined, the data processor is configured to select one of these optimal trajectories so that the displacement of the robotic arm implies the smallest number of displacements of the motorized joint(s) of the first part possible.

The initial pose can be registered as an absolute pose in a defined reference system. For instance, the initial pose can be registered as an absolute pose in a reference system associated with the robotic arm. Alternatively, the initial pose can be registered as a relative pose of the surgical tool with respect to the region of interest. According to this alternative, the data processor is configured to, as long as the pre-alignment mode is enabled, determine the displacement of the surgical tool needed to reach its initial pose based on the determined current configuration of the robotic arm, on the stored surgical plan and on the determined relative pose of the surgical tool with respect to the region of interest.

The surgical system is operable in an interactive mode, the data processor being configured to, as long as the interactive mode is enabled:
determine the current dynamics and kinematics of the robotic arm,
receive at least one user command from the control device,
determine, based on the received user command(s) and on the current dynamics and kinematics of the robotic arm, the intended displacement of the surgical tool,
receive the information related to the determined relative pose of the surgical tool with respect to the region of interest from the tracking system,
determine a predictive pose of the surgical tool with respect to the anatomical structure based on the determined intended displacement of the surgical tool and on the received information related to the determined relative pose of the surgical tool with respect to the region of interest.
   If the predictive pose of the surgical tool with respect to the anatomical structure matches a stored pose of the region of interest, the data processor is further configured to:
determine at least one optimal trajectory of the robotic arm permitting to obtain the determined intended displacement of the surgical tool, while satisfying at least one mathematical function which defines authorized trajectories of the robotic arm,
compute and send at least one instruction(s) to at least one of the motorized joint(s) of the robotic arm, the execution of the at least one instruction(s) resulting in a displacement of the robotic arm according to the optimal trajectory.

The surgical system is operable in an interactive mode, and, as long as the interactive mode is enabled, the motorized joints of the first part of the robotic arm are locked.

Satisfying the at least one mathematical function ensures to avoid one or several of the following:
Singularities,
Collisions with objects which are distinct from the robotic arm, and which are in the surroundings of the surgical system,
Collisions between distinct links of the robotic arm,
Collisions between the surgical tool and the robotic arm,
Collisions between the robotic arm and a robot station.

Thus, when it is referred to “at least one mathematical function” in the present document, it is referred to at least one mathematical function chosen among identical limited lists of mathematical functions.

The surgical tool is deactivated as long as the hand-guiding mode or the pre-alignment mode is enabled.

The surgical system comprises exactly one control device.

The first part of the robotic arm comprises three motorized joints, two of which permitting to displace the base of the second part within a plane and a third one permitting to displace the base of the second part along an axis perpendicular to said plane, and the second part of the robotic arm comprises at least seven motorized joints.

The surgical tool is a cutting tool or a cutting guide.

The present invention also concerns a method for controlling a surgical system operable in a hand-guiding mode, the surgical system comprising
at least one robotic arm coupled with a surgical tool, the robotic arm comprising several links, two adjacent links being linked to one another by one motorized joint, the robotic arm comprising at least a first part with at least three degrees of freedom and a second part with at least six degrees of freedom, an end of the first part forming a base of the second part and the first part comprising a selective compliance assembly robot arm,
   the method comprising, as long as the hand-guiding mode is enabled, the steps of
determining current dynamics and kinematics of the robotic arm,
receiving at least one user command from the control device,
determining, based on the received user command(s) and on the current dynamics and kinematics of the robotic arm, an intended displacement of the surgical tool,
determining at least one optimal trajectory of the robotic arm permitting to obtain the determined displacement of the surgical tool, while satisfying at least one mathematical function which defines authorized trajectories of the robotic arm,
computing and sending at least one instruction(s) to at least one of the motorized joint(s) of the robotic arm, the execution of the at least one instruction(s) resulting in a displacement of the robotic arm according to the optimal trajectory,
   wherein when several optimal trajectories of the robotic arm are determined, the method comprises an additional step of selecting one of these optimal trajectories
so that the displacement of the robotic arm implies the smallest number of movements of the motorized joint(s) of the first part possible.
   The present invention further concerns a method for controlling a surgical system configured to treat a region of interest of an anatomical structure, the surgical system being operable in an interactive mode, the surgical system comprising
at least one robotic arm coupled with a surgical tool, the robotic arm comprising several links, two adjacent links being linked to one another by one motorized joint, the robotic arm comprising at least a first part with at least three degrees of freedom and a second part with at least six degrees of freedom, an end of the first part forming a base of the second part and the first part comprising a selective compliance assembly robot arm,
at least one tracking system configured to determine, at a predefined frequency, relative pose of the surgical tool with respect to the anatomical structure and with respect to the region of interest,
   the method comprising, as long as the interactive mode is enabled, the steps of
determining the current dynamics and kinematics of the robotic arm,
receiving at least one user command from the control device,
determining, based on the received user command(s) and on the current dynamics and kinematics of the robotic arm, the intended displacement of the surgical tool,
receiving an information related to the determined relative pose of the surgical tool with respect to the region of interest from the tracking system,
determining a predictive pose of the surgical tool with respect to the anatomical structure based on the determined intended displacement of the surgical tool and on the received information related to the determined relative pose of the surgical tool with respect to the region of interest.

If the predictive pose of the surgical tool with respect to the anatomical structure matches a stored pose of the region of interest, the method comprises the additional steps of:
determining at least one optimal trajectory of the robotic arm permitting to obtain the determined intended displacement of the surgical tool, while satisfying at least one mathematical function which defines authorized trajectories of the robotic arm,
computing and sending at least one instruction(s) to at least one of the motorized joint(s) of the robotic arm, the execution of the at least one instruction(s) resulting in a displacement of the robotic arm according to the optimal trajectory.

### BRIEF DESCRIPTION OF THE DRAWINGS

Further details and advantages of the invention will appear in the following description. Embodiments of the invention will be described with reference to the drawings, in which:

FIG. 1 illustrates, in a perspective view, a surgical system according to an embodiment of the invention

FIGS. 2 to 4 are diagrams schematically illustrating, respectively, the surgical system of the invention operated according to a hand-guiding mode, an alignment mode, and an interactive mode,

FIGS. 5 to 8 illustrate successive steps of a surgical plan for preparing a knee to receive a graft in a knee ligamentoplasty;

FIGS. 9 to 15 illustrate successive steps of a surgical plan for preparing a knee to receive a knee prosthesis in a total knee arthroplasty.

### DESCRIPTION OF EMBODIMENTS

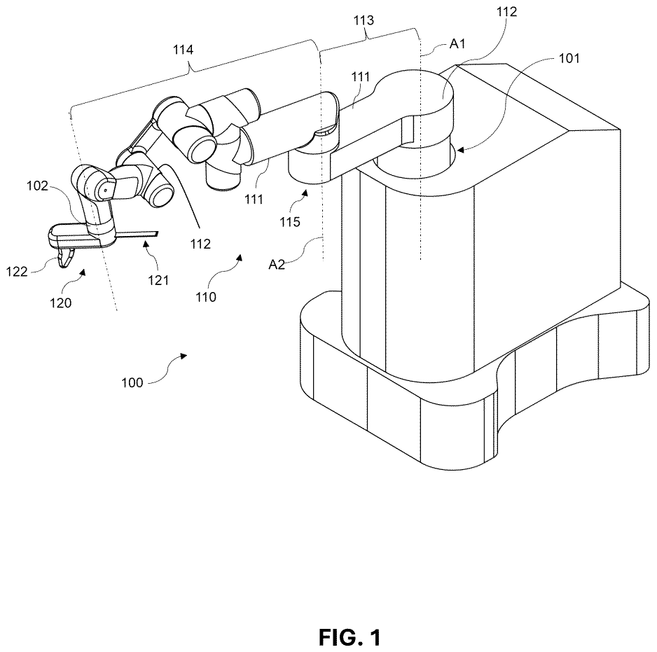

FIG. 1 illustrates, in a perspective view, a surgical system 100 according to an embodiment of the invention. In the present specification, the surgical system 100 is also referred to as “the system 100”.

The system 100 comprises at least one robotic arm 110 extending between a proximal first end 101 and a distal flange 102 to which an end-effector 120 is attached. According to the illustrated embodiment, the first end 101 is formed as a fixed cart, but the robotic arm 110 could alternatively be fixed to a wheeled cart or to a surgical table for instance, within the scope of the invention. The system 100 can also comprise a robot station—not shown here—on which can for instance be attached a display.

The end-effector 120 comprises, at least, a surgical tool 121 adapted to treat a region of interest of an anatomical structure of a patient and a control device 122. The control device 122 could be attached to any other location of the robotic arm within the scope of the invention. Alternatively, the control device could be detached from the robotic arm, without departing from the scope of the invention. The robotic arm 110 is coupled with the surgical tool 121. According to the illustrated embodiment, the surgical tool is here formed as an oscillating saw. Obviously, the surgical tool could be formed as any other kind of cutting tool, for instance a burr, a drill or a screwdriver, without departing from the scope of the invention. Alternatively, the surgical tool could also be a cutting guide adapted to guide a cutting tool. Advantageously, the end-effector 120 can be detachably attached to the flange 102 of the robotic arm 110, so that it can be changed between two successive surgeries, or during the course of one surgery if needed.

The control device 122 is configured to receive commands from a user of the system 100. As detailed below, the user of the system 100 can use the control device 122 to transmit a displacement he/she intends to apply to the surgical tool 121 to a data processor of the system 100. According to the illustrated embodiment, the control device 122 is formed as a handle coupled to a force and/or torque sensor. The control device 122, and especially the associated force and/or torque sensor, is thus configured to measure forces and/or torques applied to it by the user of the system and to determine, based on said measured forces and/or torques, a corresponding user command. As detailed below, the control device 122 is then adapted to send a corresponding information to a data processor of the system 100.

Alternatively, the control device 122 could be formed as a joystick configured to measure displacements applied to it, or as any other known input device adapted to receive commands from the user of the system 100. According to the illustrated embodiment, the system 100 comprises exactly one control device 122. Optionally, the system could comprise two or more control devices, for instance for safety reasons as if one of the control devices stops working, the user can pull the surgical tool out of the patient with the other one.

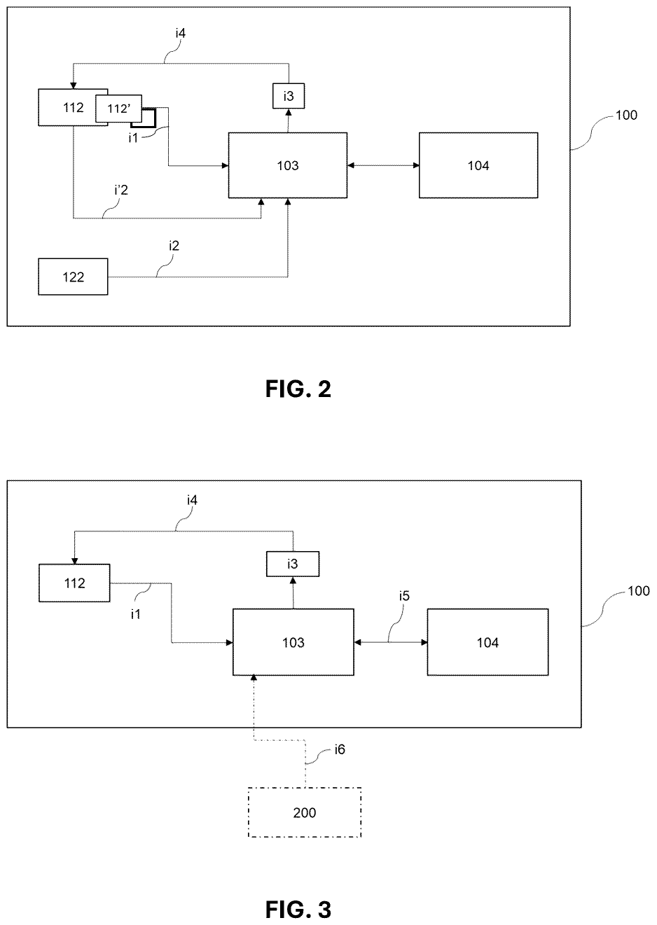

The system 100 further comprises at least one data processor 103 (also called control unit) and at least one storage unit 104—both schematically illustrated on FIG. 2. The data processor 103 is in communication with the control device 122 and with the storage unit 104. The communication can be wired or wireless within the scope of the invention. The data processor 103 and the storage unit 104 are depicted as two separate components, but they could be formed as a single unit without departing from the scope of the invention. The data processor 103 can, for instance, comprise one or more microprocessor, one or more calculators, one or more computers, one or more computer programs and/or one or more graphic processing unit. The computer program(s) comprise(s) code instructions to compute the needed instructions to be sent to motorized joints of the robotic arm, as described below. In addition, the data processor may include other devices and circuitry for performing the functions described herein such as, for example, a hard drive, input/output circuitry, and the like. The input/output circuitry can be adapted to treat digital and/or analog signals. The storage unit 104 can comprise one or more random access memory (RAM) and/or one or more read-only memory (ROM) and/or one or more non-transitory computer readable medium.

The robotic arm 110 comprises several links 111 linked to one another by motorized joints 112. The robotic arm 110 comprises a first part 113 which presents at least three degrees of freedom and a second part 114 which presents at least seven degrees of freedom, thus providing at least one redundant degree of freedom. Therefore, the robotic arm 110 comprises at least ten degrees of freedom. In the present text, the joints (and the parts) of the robotic arm are numbered in ascending order from the proximal end to the distal end.

As shown, the distal end of the first part 113 forms a proximal base 115 of the second part 114, and the distal end of the second part 114 is the flange 102 described above. The first part 113 of the robotic arm 110 is particularly designed as a SCARA (Selective Compliance Assembly Robot Arm) and thus comprises two links linked to one another by two motorized joints. As well known in the art, a SCARA comprises at least one translational degree of freedom along a first axis A1, and two axes of rotation—here referenced as A1 and A2—parallel to one another. The first part 113 of the robotic arm 110 thus permits to displace the second part 114 of the robotic arm 110, and especially the base of this second part 114, within a defined plane, perpendicular to the axes of rotation A1, A2 of the motorized joints of said first part 113. The links of the SCARA being horizontal, the defined plane is horizontal. As mentioned, this SCARA is also displaceable along a vertical axis coincident with the axis of rotation A1 of one of its motorized joints, especially coincident with the axis of rotation arranged closest to the first end 101 of the robotic arm 110.

Apart from the first joint, the other joints of the SCARA are revolute joints. More specifically, the first and second joints of the SCARA are a vertical axis prismatic joint and a vertical axis revolute joint. The first and second joints may be separate and attached end-to-end. Alternatively, the first and second joints may be merged into a sliding pivot. The third joint is a vertical axis revolute joint.

The SCARA has a bottom-up architecture, meaning that, for each pair of links, a distal link is above a proximal link. In this way, the base of the SCARA, which is the most voluminous part of the robotic arm, is located below or at substantially the same height as the operation table and does not hinder the user's gestures or risk colliding with the user or the environment.

The joints of the SCARA comprise brakes that can be activated by the control unit when it is required that the SCARA remains static.

The second part of the robotic arm can be seen as comprising three portions. By analogy with a human arm, the second part includes, from its proximal to its distal end, a “shoulder” with three revolute joints (first to third joints of the intermediate arm), an “elbow” with one revolute joint (fourth joint of the intermediate arm), and a “wrist” with three revolute joints (fifth to seventh joints of the arm portion). Various tools 10 may be attached to the last link of the wrist, such as a sagittal saw, a burr, a screwdriver, or a drill. Alternatively, the tool 10 could be replaced by a guide, itself adapted to receive and guide a portable tool handheld by a surgeon.

The first, second and third joints of the shoulder are in parallel-perpendicular-perpendicular relationship, meaning that the axis of the first joint is parallel to the axis of the third joint of the SCARA, the axis of the second joint is perpendicular to the axis of the first joint, and the axis of the third joint is perpendicular to the axis of the second joint.

The fourth joint of the second part, i.e., the elbow, is arranged in parallel relationship with the third joint.

The three motorized joints of the wrist have a perpendicular-perpendicular-perpendicular relationship such that the axes of said three revolute motorized joints are concurrent.

The particular architecture of the second part achieves redundancy, since the number of joints is greater than the number of degrees of freedom required for the movements. Such redundancy may be used both for avoiding singularities and for offering the user an unobstructed view. A robot singularity is a configuration in which the robot end-effector cannot move linearly in certain directions-in such a case, redundancy allows choosing other configurations that achieve the trajectory to reach the required tool pose. If a given configuration of the robot would obstruct the user's view, another redundant configuration may be chosen that offers an unobstructed view. An optimized starting configuration, with a well-chosen redundancy parameter, allows a greater extent of possible motion along a translation or in a plane without encountering a singularity. Redundancy also helps preventing collision risks.

A redundant joint may in practice be chosen among joints the first, second or fifth joints of the second part. If no redundancy is required, the corresponding joint may be locked during the procedure, which would improve the stiffness of the robotic arm.

Thanks to the range of movements achievable by the configuration of the joints, the robotic arm needs not be displaced on the floor during a large extent surgical procedure (like an operation of the spine) or even between different surgical procedures.

As will be illustrated later, for each surgical procedure, the SCARA may move only in a coarse “approach” phase, after which the joints of the SCARA may remain static so that only the joints of the second part of the robotic arm move for performing the surgical procedure. Although the SCARA could be moved during the surgical procedure, this may not be preferred in some cases, because, given the larger size of the joints and links, the position feedback of the joints may be less accurate.

Thanks to the architecture of the wrist, the second part of the robotic arm has an inverse kinematic solution, i.e. a set of angular positions can be directly calculated for the joints for any desired pose of the surgical tool, once the joints of the SCARA have assumed a given configuration.

Inverse kinematics is a well-known concept in the field of robotics. More detailed information may be found in robotics textbooks, such as Medical Robotics, A. Schweikard, F. Ernst, Springer International Publishing Switzerland 2015, DOI 10.1007/978-3 -319-22891-4_3 (see Chapter 3. Robot kinematics)]. Basically, the spherical architecture of the wrist (due to the fact that the three axes of the wrist are concurrent) allows simplifying the equations establishing the relationship between the pose of the surgical tool and the position of each joint of the robotic arm, without requiring approximations.

Optionally, each motorized joint 112 of the robotic arm 110 comprises at least one encoder 112′ (see FIG. 2) configured to determine, at any time, a current configuration of the concerned motorized joint. The data processor 103 is also in communication—whether wired or wireless—with the motorized joints and especially with the motors of said motorized joints, as well as with the encoders of said motorized joints. Preferably, the encoders are absolute encoders (which measure an angular position of the joint with respect to a zero position) and not incremental encoders (which measure a change of angular position). Thus, the data processor can retrieve, at any time, from those encoders a configuration of each motorized joint and determine, based on successive retrieved configurations of the motorized joints and on known models of said robotic arm, the current dynamics and kinematics of the robotic arm. The kinematics of the robotic arm refer to all information related to interactions between velocity and positioning of robotic arm, such as the speed of the robotic arm, the speed of the surgical tool, the pose of the robotic arm and/or the pose of the surgical tool. The dynamics of the robotic arm refer to all information related to interactions between forces and accelerations applied to the robotic arm, such as the acceleration of the robotic arm, the acceleration of the surgical tool, inertial forces applied to the robotic arm and/or applied to the surgical tool, and/or gravitational force applied to the robotic arm and/or applied to the surgical tool.

Obviously, here is only described one example of how to determine the current dynamics and kinematics of the robotic arm, but other known technics and/or device(s) could be used within the scope of the invention. For instance, the surgical tool could be equipped with an accelerometer, or the data processor could additionally use values of current used by the motors of the motorized joints etc. . . . .

The storage unit 104 stores at least a surgical plan which is renewed at the beginning of each new surgery. The surgical plan defines at least one initial pose of the surgical tool 121 allowing a user of the system to start treating the region of interest. The surgical plan also defines the relative pose of the region of interest with respect to the anatomical structure. Advantageously, the surgical plan comprises all the successive steps to be performed by the user of the system to treat said region of interest. The surgical plan is thus different for each surgery, and for each patient and can also be modified during an ongoing surgery. Additionally, the surgical plan can also be adapted to the preferences of the user of the system. Optionally, the surgical plan can define several initial poses of the surgical tool, each initial pose corresponding to the beginning of a particular step of the surgery. For instance, as detailed below, if the surgery consists in drilling one tunnel in a tibial bone and one tunnel in a femoral bone to prepare said bones to receive a ligament graft, the surgical plan can comprise two initial poses permitting, respectively, to drill the tibial tunnel and the femoral tunnel.

The system 100 can be associated with a tracking system configured to determine the relative poses of the surgical tool 121 with respect to the anatomical structure and with respect to the region of interest to be treated and to send a corresponding information to the data processor.

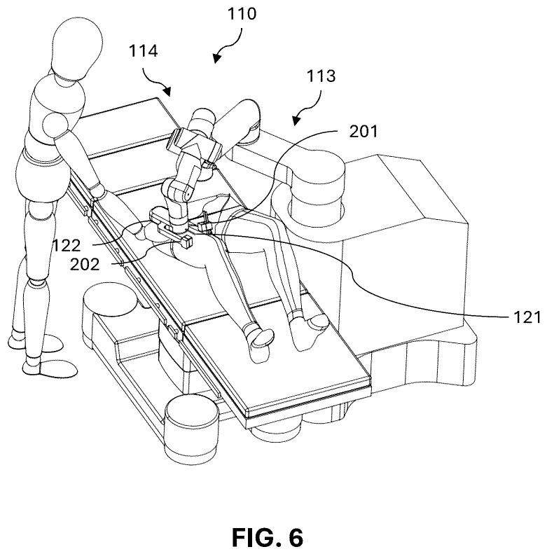

To determine the pose of the surgical tool 121 with respect to the region of interest, the tracking system can for instance be configured to first determine the relative pose of the surgical tool with respect to the anatomical structure. Then, as the pose of the region of interest with respect to the anatomical structure is known, the tracking system is configured to determine the relative pose of the surgical tool with respect to the region of interest, based on the determined pose of the surgical tool with respect to the anatomical structure and on the known pose of the region of interest with respect to the anatomical structure. Alternatively, the tracking system can be configured to determine the relative pose of the surgical tool with respect to the anatomical structure and to send a corresponding information to the data processor, the data processor then being configured to determine the pose of the surgical tool with respect to the region of interest. The tracking system can be an optical tracking system. An optical tracking system comprises optical trackers and at least one detecting device, for instance a stereoscopic camera. The optical trackers comprise a plurality of fiducials detectable by the detecting device and arranged according to a known geometry. For example, the fiducials may be reflective spheres, reflective disks, or LEDs. The tracking system is configured to determine the current poses of the optical trackers detected by the detecting device and, to compute, consequently, the respective poses of the tracked objects, such as the region of interest and/or the surgical tool. Alternatively, the tracking system could be an electromagnetic tracking system. An electromagnetic tracking system comprises at least two electromagnetic transducers including a plurality of electromagnetic coils arranged according to a known geometry. At least one electromagnetic transducer is a transmitter (also called emitter) adapted to emit at least one electromagnetic field, and at least one electromagnetic transducer is a receiver adapted to generate a response to the electromagnetic field via the generation of eddy currents in the coils of the receiver by the electromagnetic field. The tracking system is then configured to determine the relative poses of the electromagnetic transducers with respect to each other and, consequently, to compute the relative poses of the objects to which they are attached, here formed as the surgical tool and the region of interest, based on the measurements of the electromagnetic field. An embodiment of an electromagnetic tracking system is illustrated in FIG. 6.

Obviously, these are merely examples of tracking system and such a tracking system could comprise other technologies, such as accelerometers, or inertial sensors, or a combination of the above-cited technologies without departing from the scope of the invention. The tracking system could also be part of the surgical system within the scope of the invention.

The surgical system is operable according to at least three distinct modes schematically illustrated by the diagrams shown on FIGS. 2 to 4:
a hand-guiding mode, wherein the user can control movement of the robotic arm using the control device;
an alignment mode, wherein the robotic arm moves the surgical tool to an initial pose from which surgery can start; and
a patient servoing mode, wherein the robotic arm maintains a pose of the surgical tool in a coordinate system of the patient; in this mode, the robotic arm can thus maintain the surgical tool aligned with the region of interest according to the surgical plan; as a result, if the patient moves, for example due to breathing, the robotic arm with follow the movement to still have the surgical tool aligned with the region of interest.

In an embodiment, this patient servoing mode includes an interactive mode wherein the user provides an input to the robotic arm to change the pose of the surgical tool in the patient's coordinate system, in order to move the surgical tool relative to the region of interest according to the surgical plan.

In an embodiment, this patient servoing mode includes an active mode, wherein the robotic arm autonomously moves the surgical tool to execute the surgical plan.

The patient servoing mode may advantageously comprise both the interactive mode and the active mode.

Transition between any modes may be made in various ways. For example, the control device and/or the robot station may comprise one or more buttons allowing switching between the modes. Alternatively, the system may comprise a pedal coupled to the control unit, allowing the user to press the pedal with his/her foot to switch from one mode to another one.

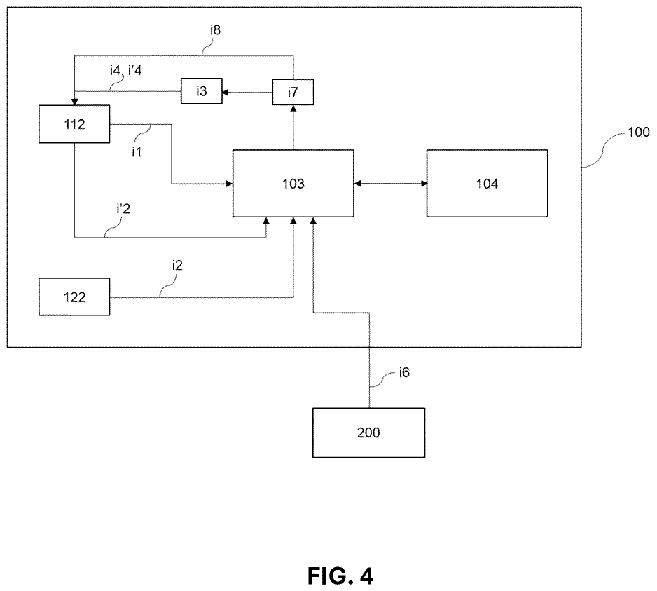

FIG. 2 illustrates a diagram representing a hand-guiding mode of the surgical system, FIG. 3 illustrates a diagram representing an alignment mode of the surgical system 100, and FIG. 4 illustrates a diagram representing an interactive mode of the surgical system 100.

When the hand-guiding mode is enabled, the robotic arm 110, and consequently the surgical tool coupled to this robotic arm, is displaced, at least partially, based on user commands received through the control device 122 described above.

Especially, the data processor 103 is configured to, as long as the hand-guiding mode is enabled:
determine the current dynamics and kinematics of the robotic arm 110. As described above, the current dynamics and kinematics of the robotic arm 110 can for instance be determined based, at least, on the information i1 received from the encoders 112′ of the motorized joints 112,
receive at least one user command i2 from the control device 122,
determine, based on the received user command(s) i2 and on the determined current dynamics and kinematics of the robotic arm 110, an intended displacement of the surgical tool 121,
determine at least one optimal trajectory i3 of the robotic arm 110 permitting to obtain the determined intended displacement of the surgical tool 121, while satisfying at least one mathematical function which defines authorized trajectories of the robotic arm 110,
compute and send at least one instruction(s) i4 to at least one of the motorized joint(s) 112 of the robotic arm 110, the execution of the at least one instruction(s) resulting in a displacement of the robotic arm 110 according to the optimal trajectory i3.

According to the invention, when several optimal trajectories of the robotic arm 110 are determined, the data processor 103 is configured to select one of these optimal trajectories so that the displacement of the robotic arm 110 implies the smallest number of movements of the motorized joint(s) 112 of the first part possible 113. A “trajectory of the robotic arm” is defined as successive movements of the motorized joints of this robotic arm. By “select one of the optimal trajectories”, we here mean that the data processor 103 is configured to choose one optimal trajectory, among the ones determined as such, for which the corresponding instructions are going to be computed.

By “intended displacement of the surgical tool”, we here refer to a displacement the user of the system wants to apply to said surgical tool. As evoked above, when the hand-guiding mode or the interactive mode is enabled, the user can transmit his/her intention concerning the displacement of the surgical tool through the control device.

For instance, the at least one mathematical function can be stored in the storage unit 104. This mathematical function is defined as a function which, when satisfied, ensures that the corresponding trajectory of the robotic arm 110 avoids, at least the following issues: singularities, collisions between the robotic arm 110 and its surroundings, collisions between the surgical tool and the robotic arm and/or collisions between the different segments of the robotic arm. Those issues can be avoided by satisfying several distinct mathematical functions, each of them permitting to avoid at least one of said issues. Alternatively, these distinct mathematical functions could be merged into one unique mathematical function. The mathematical function can also comprise parameters selected by the user of the system, such as preferred trajectories of some motorized joints for instance.

These mathematical function(s) are well known by the skilled man of the art and can for instance comprise the Gilbert Johnson Keerti function, an analytic method of collision resolution and/or a sequential method of collision resolution etc. . . . .

The computation of the optimal trajectory may also take into account predetermined parameters. For example, a predetermined parameter can include an angle of a link of the robotic arm relative to a fully extended configuration of the robotic arm, or a distance between the center of the wrist and the shoulder Indeed, some configurations, such as a full extension of the robotic arm, an alignment between the wrist and the shoulder or a position of a motorized joint close to its limit position should generally be avoided since they limit the range of movement of the robotic arm. Another (additional or alternative) predetermined parameter is a dexterity index of the robotic arm. Indeed, a dexterity index falling below a given threshold should be avoided since it could generate quick and hazardous movements of the robotic arm.

Advantageously, the data processor 103 can be configured to detect and/or measure a force and/or torque i′2 applied on at least one of the motorized joints 112 of the second part 114 of the robotic arm 110. For instance, each motorized joint 112 can comprise one forces and/or torques sensor configured to measure the forces and/or torques applied to the concerned motorized joint and to send a corresponding information to the data processor. Alternatively, each motorized joint can comprise two encoders respectively arranged before and after a reductor of the concerned motorized joint, the data processor being configured to receive values measured by said encoders and to compare them in order to determine the forces and/or torques applied to the corresponding motorized joint. According to another alternative, the data processor 103 can receive an information related to the current used by the motor of the concerned motorized joint and to determine, based on this information, on the command sent to the motor and on known parameters of said motor, the force and/or torque applied to the motorized joint. Thus, the data processor 103 can be further configured to, as long as the hand-guiding mode is enabled:
determine, based on the detected and/or measured force and/or torque i′2 applied on the concerned motorized joint(s), a configuration of the concerned motorized joint(s) intended by the user,
select the optimal trajectory i3 of the robotic arm so that the displacement of the concerned motorized joint(s) matches the intended configuration of said motorized joint(s).

Thus, when the hand-guiding mode is enabled, the system of the present invention permits the user to restrain the number of optimal trajectories between which the data processor must choose. Otherwise said, the user can physically lead, at least partially, the selection of the optimal trajectory.

FIG. 3 illustrates another mode referred to as “alignment mode”. According to the invention, the data processor 103 is configured to, as long as the alignment mode is enabled:
determine the current configuration of the robotic arm 110, thanks, for instance, to the information i1 transmitted by the encoders of each motorized joint 112
determine, based on the surgical plan i5 stored in the storage unit 104 and on the current configuration of the robotic arm, a displacement of the surgical tool needed to reach its initial pose,
determine at least one optimal trajectory of the robotic arm permitting to obtain the determined displacement of the surgical tool, while satisfying the at least one mathematical function which defines authorized trajectories of the robotic arm,
compute and send at least one instruction(s) i4 to at least one of the motorized joint(s) 112 of the robotic arm 110, the execution of the at least one instruction(s) i4 resulting in a displacement of the robotic arm 110 according to the optimal trajectory,
   wherein when several optimal trajectories of the robotic arm 110 are determined, the data processor 103 is configured to select one of these optimal trajectories so that the displacement of the robotic arm 110 implies the smallest number of displacements of the motorized joint(s) 112 of the first part 113 possible.

The initial pose of the surgical tool 121 can be an absolute pose in a defined reference system, or it can be a relative pose with respect to the region of interest. When the initial pose of the surgical tool is registered as a relative pose, the data processor is configured to receive an information i6, from the tracking system 200, related to the current pose of the surgical tool with respect to the region of interest and to use this information i6 concurrently with the stored initial pose registered with the surgical plan i5 and with the determined current configuration of the robotic arm to determine the displacement needed for the surgical tool to reach said relative initial pose. It is understood that as long as the alignment mode is enabled, the user cannot interfere in the selection of the optimal trajectory, nor in the determination of the displacement of the surgical tool.

As described with more details below, the alignment mode is activated before the beginning of the treatment, to position the surgical tool in a pose permitting to perform said treatment of the region of interest. Thus, the robotic arm is static when the alignment mode is activated, and the optimal trajectory is computed only once whereas the optimal trajectory is updated continuously, i.e. at a predetermined frequency, as long as the hand-guiding mode is enabled. The current configuration of the robotic arm that the data processor uses to determine the displacement of the surgical tool needed to reach the initial pose thus comprises poses of each motorized joint at a given time, in this case before beginning the treatment, and more particularly when the alignment mode is enabled.

Additionally, the surgical system 100 can also be operated according to an interactive mode which is part of the patient servoing mode.

In the patient servoing mode, the data processor is configured to, as long as said patient servoing mode is enabled:
determine the current dynamics and kinematics of the robotic arm, based at least on information received from the encoders of the motorized joints 112 as described above,
receive information related to the determined relative pose of the surgical tool with respect to the region of interest, from the tracking system 200,
determine at least one satisfactory trajectory of the robotic arm permitting to maintain the alignment of the surgical tool with the region of interest while satisfying at least one mathematical function which defines authorized trajectories of the robotic arm,
compute and send at least one instruction(s) to at least one of the motorized joint(s) of the robotic arm, the execution of the at least one instruction(s) resulting in a displacement of the robotic arm according to the determined trajectory.

In the interactive mode, the system remains in the patient servoing mode and further receives at least one input from the user regarding an intended displacement of the surgical tool.

FIG. 4 illustrates, schematically, the interactive mode. According to the invention, the data processor 103 is configured to, as long as the interactive mode is enabled:
determine the current dynamics and kinematics of the robotic arm, based, at least, on the information i1, for instance, received from the encoders of the motorized joints 112 as described above,
receive at least one user command i2 from the control device 122,
determine, based on the received user command(s) i2 and on the current dynamics and kinematics of the robotic arm, the intended displacement of the surgical tool,
receive the information i6 related to the determined relative pose of the surgical tool with respect to the region of interest, from the tracking system 200,
determine a predictive pose i7 of the surgical tool with respect to the anatomical structure based on the determined displacement of the surgical tool and on the received information i6 related to the determined relative pose of the surgical tool with respect to the region of interest.
   Then, if the predictive pose i7 of the surgical tool with respect to the anatomical structure matches a pose of the region of interest as stored in the surgical plan in the storage unit 104, the data processor 103 is configured to:
determine at least one optimal trajectory i3 of the robotic arm permitting to obtain the determined displacement of the surgical tool, while satisfying at least one mathematical function which defines authorized trajectories of the robotic arm,
compute and send at least one instruction(s) i4 to at least one of the motorized joint(s) of the robotic arm, the execution of the at least one instruction(s) resulting in a displacement of the robotic arm according to the optimal trajectory.

If the predictive pose i7 of the surgical tool with respect to the anatomical structure does not match any pose of the region of interest as stored in the surgical plan in the storage unit 104, the data processor 103 can then be configured to send an instruction i8 to brake the motorized joints 112 as a safety measure. Alternatively, the data processor 103 can be configured to adapt the user command for the surgical tool to remain within the region of interest once the instruction(s) i'4 are executed.

The interactive mode is a mode wherein the user of the system can transmit his/her intention of displacement of the surgical tool, permitting to treat the region of interest, but limited with safety parameter, especially, it is understood from what have been described that the surgical system is configured to comply with the intention of the user only if such intention is coherent with the stored surgical plan.

Optionally, the first part of the robotic arm can remain static as long as the patient servoing mode is enabled. To that end, the control unit may send instructions to the motors of the joints of the first part to maintain their current position. Alternatively, the control unit may stop feeding the motors of the joints of the first part with an electrical current during activation of the interactive mode, thereby locking said joints by activating brakes. In this way, the first part-which is the most voluminous-does not move when the robotic arm is in the interactive mode. As a small movement of the SCARA may result in a large displacement of the distal end of the robotic arm, imposing the SCARA to remain static allows reducing the risk of collisions with the patient, a user or the environment. In this mode, only the intermediate part and the wrist, which are more compact, can move to displace the surgical tool to perform the planned surgery.

Optionally, the surgical tool can be deactivated as long as one of the alignment mode or the hand-guiding mode is enabled.

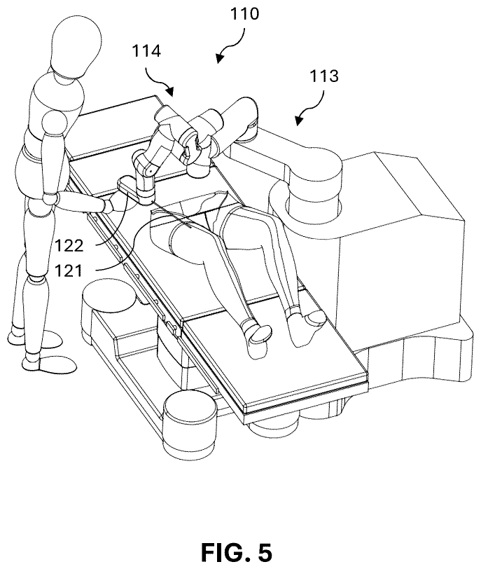

With reference to FIGS. 5 to 8 we are now going to describe the first steps to be carried to perform a knee ligamentoplasty with the surgical system 100 of the invention. As well known in the art, a knee ligamentoplasty must be begun by drilling tunnels adapted to receive the future new ligament-also referred to as a “graft”. For replacing an anterior cruciate ligament as illustrated on these figures, at least two tunnels must be drilled, respectively, in the femur and in the tibia. As schematically illustrated on FIGS. 5 to 8, to perform such a surgery the surgical tool 121 can for instance be a drill. A surgical plan for this kind of surgery thus typically comprises, at least, the respective poses of the tunnels to be drilled with respect to the concerned bones, which form the region of interest to be treated and, consequently, the respective poses of the drill forming the trajectory to drill said tunnels.

FIG. 5 illustrates a first step wherein the surgical system 100 is operated according to the alignment mode described above with reference to FIG. 3. As detailed above, the robotic arm 110 is thus configured to position, automatically, based on the stored surgical plan and on the information given by the tracking system related to the relative pose of the surgical tool with respect to the region of interest, the surgical tool 121 according to an initial pose registered in the surgical plan and permitting the user to start drilling the femoral tunnel. As previously described, this positioning is advantageously realized according to the selected optimal trajectory of the robotic arm 110. For safety reasons, the surgical system comprises an activation device, such as a button or a pedal for instance, which must be continuously set off by the user of the system to activate the alignment mode.

Once the surgical drill is positioned, the surgical system 100 is switched to be operated according to the interactive mode, for instance described above with reference to FIG. 4, wherein the user can transmit his/her commands through the control device 122 in order to drill the femoral tunnel, as illustrated on FIG. 6. It can be seen that, between the modes illustrated in FIGS. 5 and 6, the first part 113 has not moved but only the second part has moved to align the surgical tool with the femur. Indeed, the second part benefits from a sufficient amplitude of movement to drill a deep hole in the femur. In fact, the first part 113 remains in the same position as long as the interactive mode is enabled. To that end, the control unit may either stop feeding the motors of the joints of the SCARA with an electrical current and activate the brakes or send instructions to the joints of the SCARA to maintain their current position. As evoked previously, as a tracking system is used, the data processor is configured to ensure that the commands of the user comply with the surgical plan so as to ensure that the drilled tunnel is drilled where planned, i.e. at the location of the region of interest. In FIG. 6, an electromagnetic localization system is illustrated. Said system comprises a first electromagnetic transducer 201 rigidly attached to the femur and a second electromagnetic transducer 202 rigidly attached to the distal end of the robotic arm or to the surgical tool. At least one of these first and second electromagnetic transducers is an emitter and the other one is a receiver. Alternatively, both first and second electromagnetic transducers may be receivers and a third electromagnetic transducer (not shown) located in the vicinity of the first and second electromagnetic transducers is an emitter. To improve legibility of the drawings, the electromagnetic localization system has been omitted on the other figures.

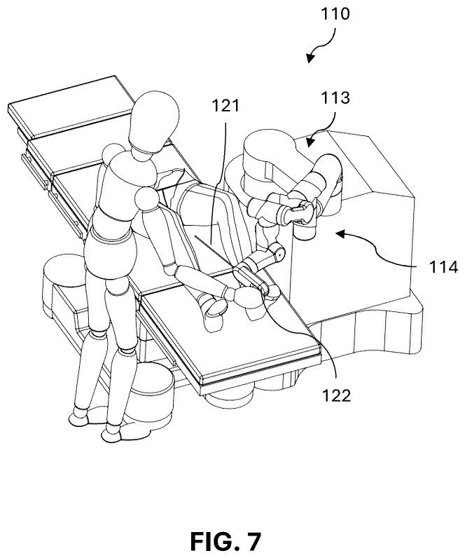

As partially illustrated on FIG. 7, the user can then pull the drill out of the patient and switch the surgical system back to its alignment mode so that the surgical drill can be positioned, automatically according to the selected optimal trajectory, in the next initial pose, i.e. the stored pose from which the user can start drilling the tibial tunnel. As can be seen from FIG. 6 to 7, when the alignment mode is enabled, the motorized joints 112 of the first part 113 move to place the second part in a more suitable pose for drilling the tibial tunnel. Indeed, the second part would not have a sufficient amplitude of movement to drill both the tibial tunnel and the femoral tunnel in the same pose of its proximal end. The SCARA thus increases the workspace of the robotic arm.

Optionally, the user can switch to the hand-guiding mode, as described with reference to FIG. 2 after having pulled the drill out of the patient to bring said tool closer to the next initial pose and only then switch to the alignment mode.

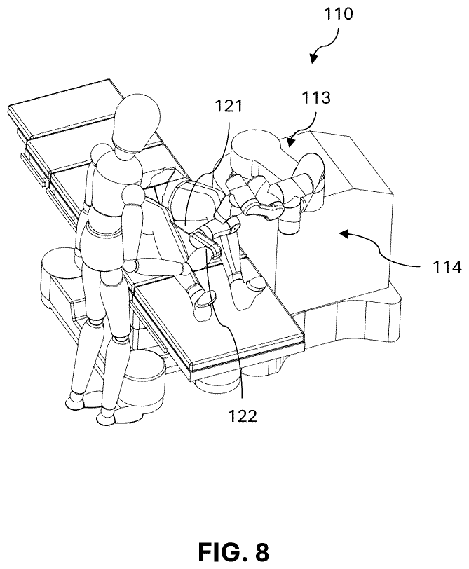

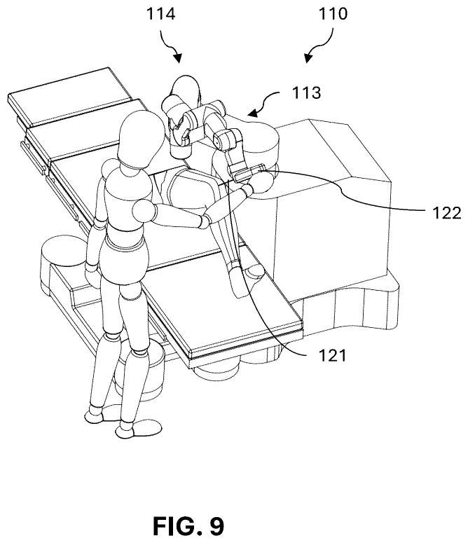

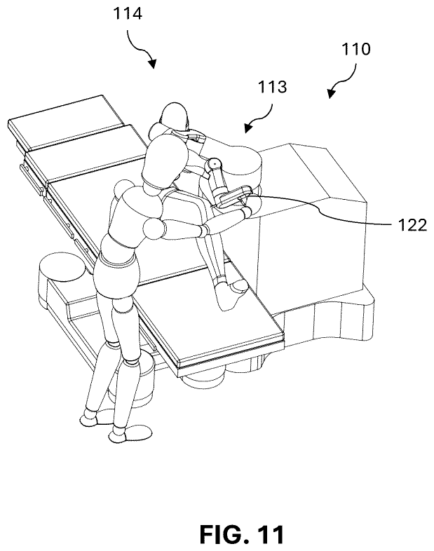

Again, once the surgical drill is positioned, the surgical system 100 can be switched to be operated according to the interactive mode wherein the user can transmit his/her commands through the control device 122 in order to drill the tibial tunnel, as illustrated on FIG. 8. Again, according to the example shown, the SCARA may remain static as long as the interactive mode is enabled. Advantageously, as a tracking system is used, the data processor is further configured to ensure that the commands of the user comply with the surgical plan so as to ensure that the drilled tunnel is drilled where planned. The user of the system can thus use the created tunnels to position correctly the new ligament. Now referring to FIGS. 9 to 14 the first steps needed to be carried to perform a total knee arthroplasty with the surgical system 100 of the invention are illustrated. Generally speaking, a total knee arthroplasty consists in implanting a knee prosthesis to help a patient recover motility of his/her knee, for instance when cartilaginous tissues have been eroded. As well known in the art, a total knee arthroplasty must be begun by machining the femur and the tibia, and especially a distal end of the femur and a proximal end of the tibia, so that the knee prosthesis can be implanted on these bones. As illustrated, to perform such a surgery the surgical tool 121 can for instance be an oscillating saw permitting to realize planar cuts. A surgical plan for this kind of surgery thus typically comprises, at least, the respective poses of six cutting planes, one of which permitting to prepare the tibia to receive a tibial component of the knee prosthesis and five of which permitting to prepare the femur to receive a femoral component of said knee prosthesis. Those cutting planes thus form the region of interest to be treated and, consequently, the respective poses of the oscillating saw forming the trajectory to machine said bones.

FIG. 9 illustrates the surgical system 100 operated according to the alignment mode described above with reference to FIG. 3. As detailed above, the robotic arm 110 is thus configured to position, automatically, based on the stored surgical plan and the information given by the tracking system related to the relative pose of the surgical tool with respect to the region of interest, the surgical tool 121 according to an initial pose registered in the surgical plan and permitting the user to start machining the tibial cut. As previously described, this positioning is advantageously realized according to the selected optimal trajectory of the robotic arm 110. For safety reasons, the surgical system of the invention comprises an activation device, such as a button or a pedal for instance, which must be continuously set off by the user of the system to activate the alignment mode.

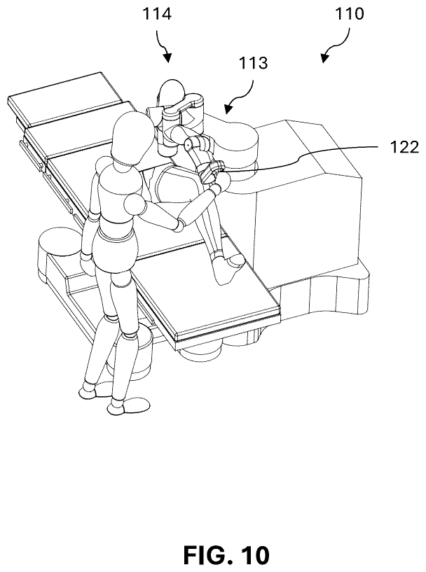

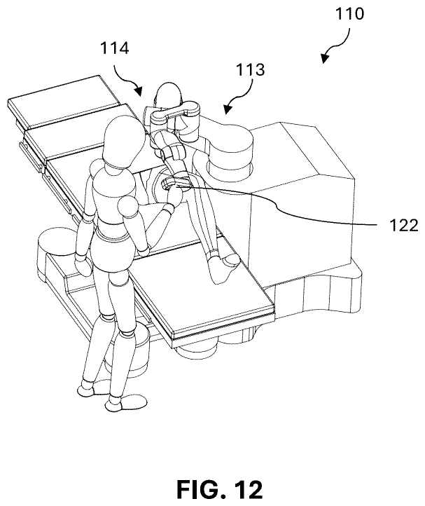

Once the oscillating saw is positioned, the surgical system 100 is switched to be operated according to the interactive mode, for instance described above with reference to FIG. 4, wherein the user can transmit his/her commands through the control device 122 in order to cut the tibial bone, as illustrated on FIGS. 10 to 12. According to the example shown, the first part 113 remains static as long as the interactive mode is enabled. Thus, this first part 113 must be in a configuration permitting the surgical tool 121 to reach the entire surface of the tibia to be cut only by moving the motorized joints of the second part 114. In particular, it can be seen that the saw can be used to cut the external side (FIG. 10) and the inner side (FIG. 12) of the tibia with the same configuration of the SCARA. As evoked previously, as a tracking system is used, the data processor is configured to ensure that the commands of the user comply with the surgical plan so as to ensure that the cut is only performed where planned.

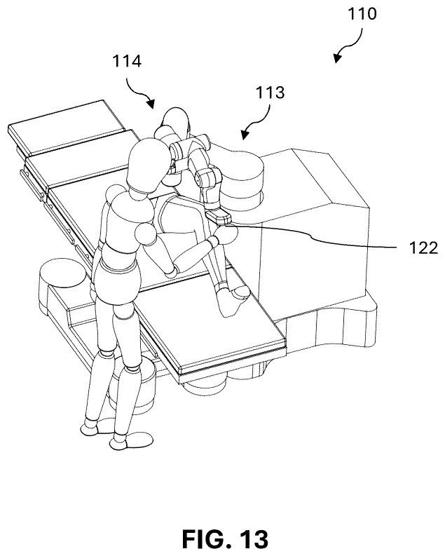

Once the tibial cut is completed, the user of the system can start the five femoral cuts, only the three first femoral cuts being illustrated, respectively, on FIGS. 13, 14 and 15. To permit the alignment of the surgical tool 121, especially the oscillating saw according to the particular example described, the user can switch the surgical system 100 from the interactive mode to the alignment mode, as described with reference to FIG. 3. As illustrated, the configuration of the first part 113 can be maintained between the tibial cut and the first femoral cut. Thus, only the second part 114 is displaced to align the surgical tool 121 with the plane of the first femoral cut. The user can then switch the surgical system back to the interactive mode to realize said first femoral cut thanks to the control device 122.

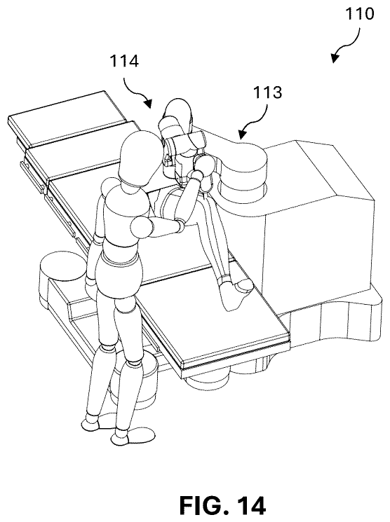

Once the first femoral cut is completed, the user of the system can start the second femoral cut for instance illustrated on FIG. 14. Again, to permit the alignment of the surgical tool 121, especially the oscillating saw according to the particular example described, the user can switch the surgical system 100 from the interactive mode to the alignment mode, as described with reference to FIG. 3. As illustrated, the configuration of the first part 113 can be maintained between the first and the second femoral cut. Thus, only the second part 114 is displaced to align the surgical tool 121 with the plane of the second femoral cut. The user can then switch the surgical system back to the interactive mode to realize said second femoral cut thanks to the control device.

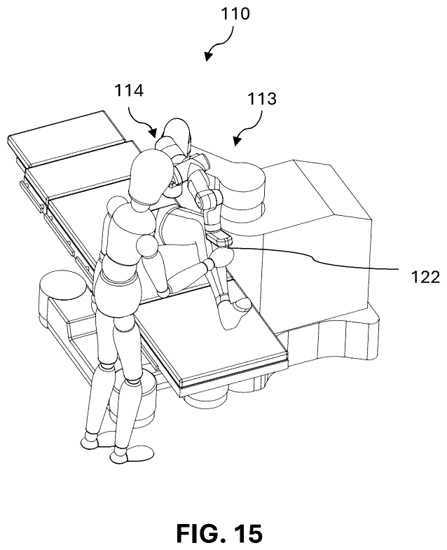

Once the second femoral cut is completed, the user of the system can start the third femoral cut, for instance illustrated on FIG. 15. To permit the alignment of the surgical tool, especially the oscillating saw according to the particular example described, the user can switch the surgical system 100 from the interactive mode to the alignment mode, as described with reference to FIG. 3. As illustrated, the configuration of the first part 113 is modified between the second femoral cut and the third femoral cut, which is allowed, as long as the alignment mode is enabled. Thus, both the first part 113 and the second part 114 are displaced to align the surgical tool with the plane of the third femoral cut. The user can then switch the surgical system back to the interactive mode to realize said third femoral cut thanks to the control device 122.

The described steps can then be repeated as many times as needed to realize all five femoral cuts.

As shown in FIGS. 5 to 14, the robotic arm may be placed on the side of the operation table opposite the user. In this way, the risks of collision between the robotic arm and the user are minimized, and the user benefits from a wide space around him/her. In some situations, the user may be on the same side of the operation table as the robotic arm. In such cases, as the most voluminous part of the robotic arm (which is the base of the SCARA) constitutes the lower part of the robotic arm, the space surrounding the arms and head of the user is mainly accessible by the second part of the robotic arm, which is lighter and less voluminous than the SCARA.

Obviously, those are only examples of applications for which the surgical system of the invention can be used, which does not restrict the invention. The surgical system could be used to perform many other kinds of surgeries, and particularly orthopedic surgeries, without departing from the scope of the invention.

### REFERENCES

S. Lavallée & P. Cinquin: “Computer assisted medical interventions” In K. H. Hohne, editor, NATO ARW, 30 Imaging in Medicine, Vol F60, 301-312, Berlin, June 1990. Springer-Verlag
Medical Robotics, chapter 3. Robot kinematics, A. Schweikard, F. Ernst, Springer International Publishing Switzerland 2015, DOI 10.1007/978-3-319-22891-4_3

1. A surgical system comprising:
a robotic arm comprising, from a proximal to a distal end:
a SCARA comprising a plurality of parallel motorized joints connected by a plurality of links extending in a horizontal plane, wherein, for each pair of links, a distal link is above a proximal link;
an intermediate arm comprising four revolute motorized joints having a parallel-perpendicular-perpendicular-parallel relationship; and
a wrist comprising an interface for holding a surgical tool, including three revolute motorized joints having a perpendicular-perpendicular-perpendicular relationship such that axes of said three revolute motorized joints are concurrent;
at least one control device configured to receive commands from a user; and
a control unit coupled to the robotic arm and the control device to operate the robotic arm according to at least a hand-guiding mode and a patient servoing mode, the control unit being configured to:
determine a current configuration of the joints of the robotic arm;
compute a target configuration of the joints of the robotic arm to move the surgical tool based on commands received from the user and on the current configuration of the joints;
compute a trajectory of the robotic arm to achieve said computed target configuration; and
send instructions to at least one motor of the joints of the SCARA, the intermediate arm or the wrist to move the surgical tool according to the computed trajectory.

2. The surgical system of claim 1, further comprising a tracking system coupled to the control unit, the tracking system being configured to track a pose of the surgical tool with respect to at least one region of interest of an anatomical structure to be treated by the surgical tool.

3. The surgical system of claim 2, wherein the control unit comprises a storage unit storing a surgical plan defining the at least one region of interest to be treated and at least one initial pose of the surgical tool allowing a user of the system to start treating the region of interest of an anatomical structure.

4. The surgical system of claim 3, wherein the control unit is configured to operate the robotic arm in an alignment mode, wherein the control unit is configured to
compute a target configuration of the joints of the robotic arm to move the surgical tool to the at least one initial pose;
compute a trajectory of the robotic arm to achieve said computed target configuration; and
send instructions to at least one motor of the joints of the SCARA, the intermediate arm or the wrist to move the surgical tool according to the computed trajectory.

5. The surgical system of claim 1, wherein each motorized joint of the robotic arm comprises at least one encoder coupled to the control unit, the control unit being configured to determine the current configuration of the robotic arm based on information received from each encoder.

6. The surgical system of claim 1, wherein the motorized joints of the intermediate arm and the wrist are arranged to have an analytical inverse kinematic solution, whereby the control unit is configured to calculate a set of angular positions for the joints for a desired pose of the surgical tool.

7. The surgical system of claim 1, wherein the control unit is configured to identify a risk of singularity in a computed configuration of the robotic arm and determine at least one alternative configuration of the intermediate arm or the wrist to achieve a configuration of the robotic arm avoiding the singularity.

8. The surgical system of claim 3, wherein, in the patient servoing mode, the control unit is configured to update the trajectory of the robotic arm to maintain alignment of the surgical tool with the region of interest according to the surgical plan.

9. The surgical system of claim 8, wherein, in the patient servoing mode, the control unit is configured to send instructions to the joints of the SCARA to maintain their current position or lock the joints of the SCARA by stopping feeding the motors of the joints of the SCARA with an electrical current and activate brakes.

10. The surgical system of claim 1, wherein the control device is fixed to the robotic arm.

11. The surgical system of claim 1, wherein the control device is detachably mounted on the robotic arm.
